(19) United States
(12) Patent Application Publication
(10) Pub. No.: US 2018/0080846 A1
(43) Pub. Date: Mar. 22, 2018

(54) MOBILE HAZGAS/FIRE DETECTION SYSTEM

(71) Applicant: GENERAL ELECTRIC COMPANY, Schenectady, NY (US)

(72) Inventors: Hua Zhang, Greenville, SC (US); Manuel Cardenas, JR., Greenville, SC (US)

(21) Appl. No.: 15/270,645

(22) Filed: Sep. 20, 2016

(51) Int. Cl. G01M 3/04; G01M 15/14; G01N 33/00

(52) U.S. Cl. CPC G01M 3/04; G01N 33/0036; G01M 15/14

(57) ABSTRACT

A system includes a gas turbine enclosure and a robotic mobile hazardous gas detection device disposed within the gas turbine enclosure. The robotic mobile hazardous gas detection device includes one or more sensors configured to detect one or more parameters related to hazardous gas leakage within the gas turbine enclosure. The robotic mobile hazardous gas detection device is configured to move to different locations within the gas turbine enclosure to monitor for hazardous gas leakage.

### BACKGROUND

The present disclosure relates generally to gas turbines. In particular, the present disclosure relates to systems for hazardous gas leak detection in a turbine enclosure.

Gas turbines are used to generate power for various applications. To protect the turbine from the surrounding environment and vise versa, the gas turbine may be housed or enclosed in an enclosure or turbine enclosure with appropriate inlets, exhaust outlets, and ventilations, etc. For example, a gas turbine may be housed inside an enclosure, which may facilitate reducing noise during turbine operation and contain environmental hazards such as fuel gas from leaking to the surrounding environment. While sensors may be installed to monitor the general health of the fuel and piping systems and detect major fuel leaks, it sometimes requires many local sensors to meet the safety standard, and the maintenance of these sensors may not be easy. Furthermore, it is difficult to detect minor leaks and/or identify the exact situation of the leaks if the sensor is not located within proximity of the leak location. The minor leaks may go unnoticed and result in decreased productivity and reliability of the turbine system.

### BRIEF DESCRIPTION

Certain embodiments commensurate in scope with the originally claimed subject matter are summarized below. These embodiments are not intended to limit the scope of the claimed embodiments, but rather these embodiments are intended only to provide a brief summary of possible forms of the subject matter. Indeed, the presently claimed embodiments may encompass a variety of forms that may be similar to or different from the embodiments set forth below.

In a first embodiment, a system includes a gas turbine enclosure and a robotic mobile hazardous gas detection device disposed within the gas turbine enclosure. The robotic mobile hazardous gas detection device includes one or more sensors configured to detect one or more parameters related to hazardous gas leakage within the gas turbine enclosure. The robotic mobile hazardous gas detection device is configured to move to different locations within the gas turbine enclosure to monitor for hazardous gas leakage.

In a second embodiment, a robotic mobile hazardous gas detection device includes one or more sensors configured to detect one or more parameters related to hazardous gas leakage within a gas turbine enclosure, and a transmitter configured to wirelessly transmit the one or more parameters detected by the one or more sensors to a controller disposed outside of the gas turbine enclosure. The robotic mobile hazardous gas detection device also includes a receiver configured to wirelessly receive information related to monitoring for hazardous gas leakage from the controller. Furthermore, the robotic mobile hazardous gas detection device is configured to fly to different locations within the gas turbine enclosure having a gas turbine engine and to monitor for hazardous gas leakage.

In a third embodiment, a robotic mobile hazardous gas detection device includes one or more sensors configured to detect one or more parameters related to hazardous gas leakage within a gas turbine enclosure and a memory. The robotic mobile hazardous gas detection device also includes a processor configured to execute instructions stored on the memory that cause the robotic mobile hazardous gas detection device to determine if leakage of hazardous gas is occurring within a gas turbine enclosure based on the one or more parameters related to hazardous gas leakage. Furthermore, the robotic mobile hazardous gas detection device is configured to fly to different locations within the gas turbine enclosure having a gas turbine engine and to monitor for hazardous gas leakage.

### BRIEF DESCRIPTION OF THE DRAWINGS

These and other features, aspects, and advantages of the presently disclosed techniques will become better understood when the following detailed description is read with reference to the accompanying drawings in which like characters represent like parts throughout the drawings, wherein:

FIG. 1 is a partial schematic illustration of a turbine system having a gas turbine in a gas turbine enclosure, in accordance with an embodiment;

FIG. 2 is a schematic illustration of the turbine system utilizing a robotic mobile hazardous gas detection device for monitoring for gas leakage, in accordance with an embodiment of the present disclosure;

FIG. 3 is a schematic illustration of a robotic mobile hazardous gas detection device, in accordance with an embodiment of the present disclosure; and

FIG. 4 is a flow chart illustrating a method for utilizing the robotic mobile hazardous gas detection device, in accordance with an embodiment of the present disclosure.

### DETAILED DESCRIPTION

One or more specific embodiments of the presently disclosed embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the presently disclosed embodiments, the articles “a,” “an,” “the,” and “said” are intended to mean that there are one or more of the elements. The terms “comprising,” “including,” and “having” are intended to be inclusive and mean that there may be additional elements other than the listed elements.

As described below, a robotic mobile device may be developed to proactively detect hazardous gas or “hazgas” (e.g., fuel gas) leakage situation and contribute to an active hazgas monitoring system to provide system assessment of hazgas leakage situation on start. More specifically, the robotic mobile device may include one or more miniature sensors (e.g., temperature, micro-gas sensor, partial pressure sensor, camera, infrared imaging) to collect various data related to the hazgas leakage and enable identifying the exact situation of leakage (e.g., concentration, volume, location). The robotic mobile device may include an algorithm (e.g., global positioning system or GPS) and/or a model of the enclosure (e.g., lay out information of piping and equipment inside the enclosure, dimensions of the enclosure, mapping, etc.), and the robotic mobile device may move (e.g., fly) anywhere inside the enclosure. Furthermore, the robotic mobile device may connect (e.g., wirelessly send and receive signals) to the active hazgas monitoring system (e.g., a model connected to a service platform such as a cloud computing service, distributed control system, etc. to generate diagnostic/assessment reports, maintenance and repair recommendations, and operation adjustments of the turbine system) such that upon receiving control signals, the robotic mobile device may survey and/or move to specific locations inside the enclosure to collect data/information related to the hazgas leakage. The robotic mobile device is capable of detecting and determining minor hazardous leaks (e.g., leaks with low concentration or amount), and more precisely monitor the leakage situation without the need of disposing multiple local hazgas detecting sensors throughout the enclosure. Furthermore, the robotic mobile device capable of proactive leakage detection may be integrated into digital power plant, which leverages state of art sensor and connectivity as well as navigation to enhance the reliability and digitalization of the power plant.

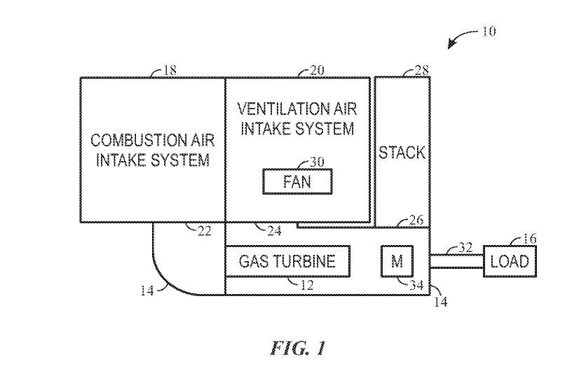

FIG. 1 is a partial schematic of an embodiment of a turbine system 10, enclosed or housed by a turbine enclosure 14 (e.g., gas turbine enclosure). The turbine system 10 may be a stationary or mobile gas turbine power generation unit. For example, the turbine system 10 may be a stationary unit disposed in a power plant, such as integrated gasification combined cycle (IGCC) power plant. For example, the turbine system 10 may be a mobile unit carried by a trailer. The turbine system 10 includes a gas turbine or gas turbine engine 12, the enclosure 14 (e.g., gas turbine enclosure) that houses the gas turbine 12, and a load 16 (e.g., generator, electrical generator) driven by the gas turbine 12. The turbine system 10 also includes a combustion air intake system 18 upstream from the gas turbine 12, and a ventilation air intake system 20. The gas turbine enclosure 14 may define a first intake port 22 (e.g., first air intake port or turbine air intake), a second intake port 24 (e.g., second air intake port or enclosure ventilation intake), and an air exit port 26.

The first intake port 22 is coupled to the combustion air intake system 18 upstream from the gas turbine 12. The combustion air intake system 18 may include one or more filters to filter air provided to the gas turbine 12. The first intake port 22 directs air into the gas turbine 12. For example, the first intake port 22 may direct air into a compressor of the gas turbine 12. For example, the gas turbine 12 may compress the air from port 22, mix the air with fuel, and combust the air-fuel mixture to drive one or more turbines. The second intake port 24 is coupled to the ventilation air intake system 20. The ventilation air intake system 20 may include one or more filters to filter air provided to the enclosure 14 of the gas turbine 12. The ventilation air intake system 20 may provide air into the enclosure 14 via one or more fans 30. The second intake port 24 directs air into the enclosure 14 surrounding the gas turbine 12 to ventilate the enclosure. The exit port 26 is coupled to an exhaust stack 28 for venting exhaust gases from the gas turbine 12 and air (e.g., ventilation air) from the enclosure 14. The gas turbine 12 includes a shaft 32 that extends through the enclosure 14 and couples to the load 16. As described in greater detail below, a robotic mobile device or robotic mobile hazardous gas detection device 34 may be utilized within the enclosure 14 for detecting, monitoring, and assessing the fuel leakage situation.

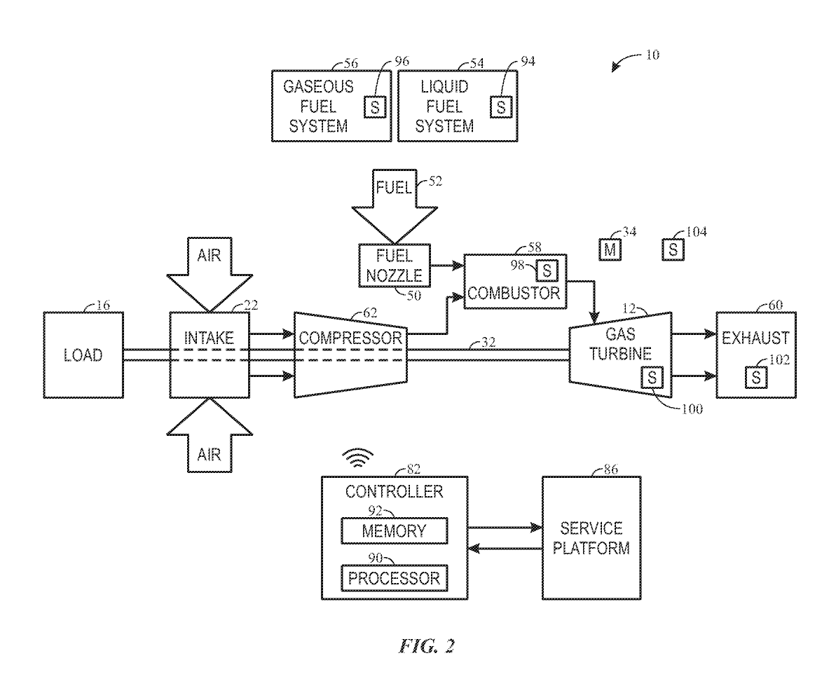

FIG. 2 is a schematic of an embodiment of the turbine system 10 utilizing the mobile device 34 for monitoring for gas leakage. The turbine system (e.g., gas turbine system, dual-fuel turbine system) 10 may use liquid or gas fuel, such as natural gas and/or a hydrogen rich synthetic gas, to drive the turbine system 10. As depicted, fuel nozzles 50 (e.g., multi-tube fuel nozzles) intake a fuel supply 52 from a liquid fuel system 54 or a gaseous fuel system 56, mix the fuel with an oxidant, such as air, oxygen, oxygen-enriched air, oxygen reduced air, or any combination thereof. Although the following discussion refers to the oxidant as the air, any suitable oxidant may be used with the disclosed embodiments. Once the fuel and air have been mixed, the fuel nozzles 50 distribute the fuel-air mixture into a plurality of combustors 58 in a suitable ratio for optimal combustion, emissions, fuel consumption, and power output. The turbine system 10 may include one or more fuel nozzles 50 located inside the plurality of combustors 58. The fuel-air mixture combusts in a chamber within each of the plurality of combustors 58, thereby creating hot pressurized exhaust gases. The plurality of combustors 58 direct the exhaust gases through the gas turbine 12 toward an exhaust outlet 60 (e.g. directed to the exit port 26). As the exhaust gases pass through the gas turbine 12, the gases force turbine blades to rotate the drive shaft 32 along an axis of the turbine system 10. As illustrated, the shaft 32 may be connected to various components of the turbine system 10, including a compressor 62. The compressor 62 also includes blades coupled to the shaft 32. As the shaft 32 rotates, the blades within the compressor 62 also rotate, thereby compressing air from the turbine air intake 22 through the compressor 62 and into the fuel nozzles 50 and/or the plurality of combustors 58. The shaft 32 may also be connected to the load 16, which may be a vehicle or a stationary load, such as an electrical generator in a power plant or a propeller on an aircraft, for example. The load 16 may include any suitable device capable of being powered by the rotational output of the turbine system 10. The fuel nozzle 52 may contain or connect with an end cover having fuel plenums, which may improve fuel distribution by feeding fuel directly into fuel injectors, which may feed fuel into tubes where it premixes with air before being released to the plurality of combustors 58. As described in greater detail below, a robotic mobile device 34 may be utilized within the enclosure 14 for detecting, monitoring, and assessing the fuel leakage situation.

A hazgas detection system may include a controller 82 of the turbine system 10 (e.g., disposed outside of the enclosure 14) and a service platform 86 (e.g., cloud computing service, distributed control system). The controller 82 is communicatively coupled (e.g., data transfer, receiving and giving instructions) with the service platform 86 and various components and systems of the turbine system 10 (e.g., gaseous fuel system 56 and liquid fuel system 54) via wired or wireless network or communication system. In some embodiments, the controller 82 may be part of the service platform 86 (e.g., cloud computer service, distributed control system, etc.). The controller 82 has a processor 90 and a memory 92 (e.g., a non-transitory computer-readable medium/memory circuitry) communicatively coupled to the processor 90, storing one or more sets of instructions (e.g., processor-executable instructions) implemented to perform operations related to the gas turbine system 10 (e.g., various components and systems of the turbine system 10). More specifically, the memory 92 may include volatile memory, such as random access memory (RAM), and/or non-volatile memory, such as read-only memory (ROM), optical drives, hard disc drives, or solid-state drives. Additionally, the processor 90 may include one or more application specific integrated circuits (ASICs), one or more field programmable gate arrays (FPGAs), one or more general purpose processors, or any combination thereof. Furthermore, the term processor is not limited to just those integrated circuits referred to in the art as processors, but broadly refers to computers, processors, microcontrollers, microcomputers, programmable logic controllers, application specific integrated circuits, and other programmable circuits.

For example, the memory 92 may store a model of the enclosure 14 (e.g., lay out information of piping and equipment inside the enclosure, dimensions of the enclosure, mapping, etc.). For example, the memory 92 may store information inputted by operators or users (e.g., via the controller 82 and/or via the service platform 86). For example, the memory 92 may store instructions as to obtain information (e.g., operational parameters and operational conditions) from various components and systems of the turbine system 10, and store the obtained information in the memory 92. The information may be collected via sensing devices inside the enclosure 14 (e.g., the robotic mobile device 34), disposed within the enclosure 14 (e.g., on any components shown in FIG. 1) and/or disposed on components of the gas turbine system 10 (e.g., on any components shown in FIG. 2). For example, these sensing devices may include the robotic mobile device 34, one or more sensors 94 of the liquid fuel system 54, one or more sensors 96 of the gaseous fuel system 56, one or more sensors 98 of the plurality of combustors 58, one or more sensors 100 of the gas turbine 12, one or more sensors 102 of the exhaust 60, and one or more sensor 104 disposed within the turbine enclosure 14. The one or more sensors 94, 96, 98, 100, 102, and 104 may include, but are not limited to temperature sensors (e.g., thermocouples, resistance temperature detectors or RTDs, and surface acoustic wave sensors or SAWs), pressure sensors (e.g., pressure transducers, pressure transmitters, piezometers, pressure indicators, and manometers), gas sensors (e.g., microstructured gas sensors, infrared point sensors, infrared cameras, ultrasonic sensors, electrochemical gas sensors, semiconductor sensors, electrochemical sensors, and calorimetric gas sensors, SAWs), flow sensors (e.g., flow meters, thermal mass flow meters, and ultrasonic flow meter), accelerometers (e.g., high temperature accelerometers), speed sensors (e.g., turbine speed sensors and magnetic speed sensors), position sensors, electrical current sensors, voltage sensors, and timers. The robotic mobile device 34 may include one or more sensors such as microstructure gas sensors (e.g., temperature cycling of semiconductor gas sensors), infrared point sensors, calorimetric thermoelectric gas sensors, electrochemical gas sensors, infrared or IR Camaro, ultrasonic sensors, surface acoustic wave sensors or SAWs, and a combination thereof. In addition, the robotic mobile device 34 may also include any sensors (e.g., types of sensors) included by the one or more sensors 94, 96, 98, 100, 102, and 104.

The one or more sensors 94, 96, 98, 100, 102, and 104, and the robotic mobile device 34 are coupled to the controller 82 to obtain the information (e.g., operational parameters and operational conditions). For example, the information may include, but not limited to enclosure air pressure and temperature, enclosure ventilation fan flow rate and fan curves, hazgas concentration, fuel gas pressure and temperature, partial pressure of the liquid fuel vapor, leakage volume, leaking rate, leakage size, leakage location, fuel gas flow rate, turbine power output and efficiency, compressor air flow rate, discharge temperature and pressure, and gas turbine exhaust temperature, etc. It may be appreciated that any of the parameters disclosed above may be determined based on time weighted average data. Furthermore, the one more sensors 94, 96, 98, 100, 102, and 104 and/or the robotic mobile device 34 within the enclosure 14 may include at least one acoustic wave sensor or surface acoustic wave sensor (SAW), which is capable of detecting the partial pressure of liquid fuel vapor and detecting a large range of gases on a single sensor with resolution down to parts per trillion.

As discussed earlier, the robotic mobile device 34 may be utilized to collect data and/or information related to hazgas leakage. It may be appreciated that the mobile device 34 may be any suitable device that is remotely or wirelessly controlled (e.g., via the controller 82) and capable of moving to different locations (e.g., flying) inside the enclosure 14 (e.g., remote controlled miniature airplane, helicopter, drum, etc.). Furthermore, the mobile device 34 may function autonomously (e.g., function without receiving external instructions). For example, the mobile device 34 may be guided by the above mentioned algorithm (e.g., GPS) and/or the model of the enclosure (e.g., lay out information of pipping and equipment inside the enclosure, dimensions of the enclosure, mapping, etc.) to automatically survey anywhere inside the enclosure 14 to monitor and collect data. For example, the mobile device 34 may also have a built-in algorithm as to detect and determine that a leakage has occurred and automatically move to the leakage location to closely monitor the leakage situation/condition.

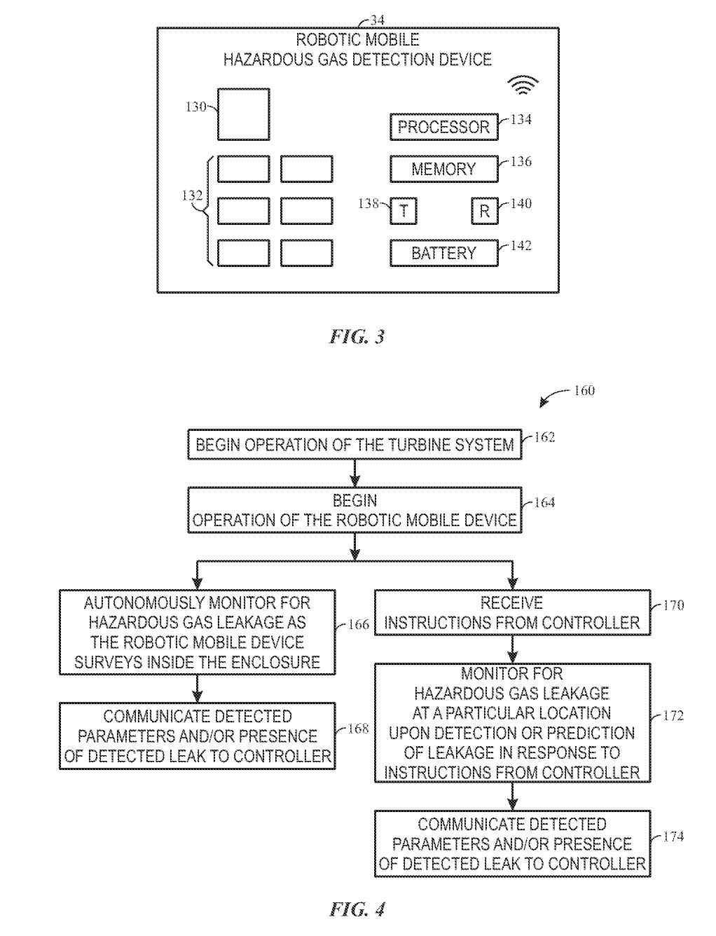

FIG. 3 is a schematic illustration of the robotic mobile device 34. The robotic mobile device 34 may have dimensions that are smaller than about 50 cm×50 cm×50 cm, about 25 cm×25 cm×25 cm, or about 10 cm×10 cm×10 cm (e.g., in length×width×height). In the illustrated embodiment, the robotic mobile device 34 may include a mobility device 130 (e.g., one or more propellers, propulsion, etc.) to enable the mobile device 34 to move (e.g., fly) to different locations, one or more sensors 132, a processor 134, and a memory 136 (e.g., a non-transitory computer-readable medium/memory circuitry) communicatively coupled to the processor 132. The robotic mobile device 34 may include a transmitter 138 and a receiver 140 (e.g., radio frequency or wireless transmitter and receiver) communicatively coupled to the processor 134 to enable the processor 134 to transmit and receive data and/or signals from the controller 82. In other embodiment, the transmitter 138 and the receiver 140 may also enable the processor 134 to transmit and receive data and/or signals from the service platform 86. The robotic mobile device 34 include a battery or energy storage device 142 to power the robotic mobile device 34. In other embodiment, the robotic mobile device 34 may be radio frequency (RF) powered or wirelessly powered.

The processor 134 may include one or more application specific integrated circuits (ASICs), one or more field programmable gate arrays (FPGAs), one or more general purpose processors, or any combination thereof. Furthermore, the term processor is not limited to just those integrated circuits referred to in the art as processors, but broadly refers to computers, processors, microcontrollers, microcomputers, programmable logic controllers, application specific integrated circuits, and other programmable circuits. The memory 136 may include volatile memory, such as random access memory (RAM), and/or non-volatile memory, such as read-only memory (ROM), optical drives, hard disc drives, or solid-state drives. The memory 136 may store one or more sets of instructions (e.g., processor-executable instructions) implemented to perform operations related to surveying and collecting data using the robotic mobile device 34.

More specifically, the memory 136 may store algorithms (e.g., global positioning system, GPS and/or other built-in algorithms to add navigation) and/or the model of the enclosure 14 (e.g., lay out information of pipping and equipment inside the enclosure, dimensions of the enclosure, mapping, etc.) such that the robotic mobile device 34 may survey (e.g., fly) anywhere inside the enclosure 14. For example, the memory 136 may store a space-based navigation system (e.g., global positioning system or GPS) that provides location, mapping, and time information. The memory 136 may store information inputted by operators or users (e.g., via the controller 82 and/or via the service platform 86). For example, the memory 136 may store instructions to survey inside the enclosure 14 along designated routes to obtain information and collect data using the one or more sensors 132. For example, the memory 136 may store instructions to obtain information and collect data (e.g., using the one or more sensors 132) at specific locations (e.g., site, component, a section of a component). For example, the memory 136 may store algorithms that may determine that hazgas leakage has occurred based on hazgas concentration and/or leakage size detected by the one or more sensors 132. Herein, the leakage size refers to volume with hazgas concentration level that falls between the lower explosive limit (LEL) and the upper explosive limit (UEL) (e.g., the amount of gas between the two limits are explosive). For example, the memory 136 may store algorithms that may update or modify the instructions to obtain information and collect data based on diagnostics or predictions. More specifically, the algorithms may determine that upon a detection of hazgas leakage, the robotic mobile device 34 may terminate its routine surveying or terminate from an idle mode (e.g., the robotic mobile device 34 is on but not collecting data) and move to the leakage location to collect data and information related to the leakage. The algorithms may also determine that upon a predication of hazgas leakage, the robotic mobile device 34 may terminate its routine surveying or terminate from an idle mode and move to the predicted location for monitoring of a relatively high risk area (e.g., location that is predicted to have a higher chance of leakage). The algorithms may determine that based on the detected leakage situation (e.g., concentration, size, location), certain sensors of the one or more sensors 132 may be activated or deactivated to collect relevant data/information. In addition, the algorithms may also determine to adjust the frequency of data collection using any of the one or more sensors 132 based on the detected leakage situation (e.g., concentration, size, location). As such the robotic mobile device 34 may have self-mobility to search within the enclosure 14 (e.g., automatically and autonomously move within the enclosure 14 and perform leakage detection).

The one or more sensors 132 may include microstructure gas sensors (e.g., temperature cycling of semiconductor gas sensors), infrared point sensors, calorimetric thermoelectric gas sensors, electrochemical gas sensors, infrared or IR Camaro, ultrasonic sensors, surface acoustic wave sensors or SAWs, and a combination thereof. The one or more sensors 132 are coupled to the processor 134 to obtain the information and/or collect data related to the hazgas leakage situation to be fed to the controller 82. For example, the information/data may include, but not limited to enclosure air pressure and temperature, hazgas concentration, partial pressure of the liquid fuel vapor, leakage volume, leaking rate, leakage size, leakage location, etc. The information and/or data collected via the one or more sensors 132 may be stored in the memory 136 and/or transmitted to the controller 82.

It may be appreciated that the robotic mobile device 34, coupled to the controller 82, may obtain information related to hazgas leakage on demand (e.g., any time) from any locations inside the enclosure 14, thus adds to the more accurate and proactive leakage monitoring. For example, if the leakage size is small and the hazgas concentration is low, a sensor (e.g., sensor disposed at a fixed location) that is far away from the leakage location may not collect adequate information/data due to the distance. In this situation, the robotic mobile device 34 is not limited to a fixed location and can move (e.g., fly) to a proximity of the leakage location to collect information/data related to the leakage situation. The robotic mobile device 34 may also be used to perform a routine surveying of the entire enclosure 14 (e.g., via one or more routes) to actively detect leakage at any locations, in addition to locations that may not easily monitored given the distribution of the fixed position sensors. In addition, the robotic mobile device 34 may collect data along its travel path and use such data to develop a profile of the detected parameter along the travel (e.g., variation or gradient profile along the travel path for enclosure air pressure and temperature, hazgas concentration, partial pressure of the liquid fuel vapor, leakage volume, leaking rate, leakage size, leakage location).

Furthermore, as set forth above, the robotic mobile device 34 may move to specific locations to collect information/data related to hazgas leakage based on diagnostics and/or predictions. For example, upon a prediction of fuel leakage conditions (e.g., concentration, rate, volume, size, and location) occurring at certain location at a future time, the robotic mobile device 34 may move to proximity of the particular location (e.g., upon receiving control signal from the controller 82 or the service platform 86) to monitor the situation (e.g., prior to, during and/or after the occurrence of leakage). It may be appreciated that the control signal from the controller 82 may be sent to the robotic mobile device 34 based on the diagnostic results and/or predictions. For example, the controller 82 may utilize a hazgas monitoring model to diagnose or predict that leakage has or will occur at certain location within the enclosure 14, and the controller 82 thus send the control signal to send the robotic mobile device 34 to that particular location to perform hazgas detection and monitoring. It may also be appreciated that since the robotic mobile device 34 is coupled to the controller 82, the robotic mobile device 34 has self-mobility to search within the enclosure 14 (e.g., automatically move within the enclosure 14 and perform leakage detection based on diagnostic results or predictions). In other embodiments, the controller 82 may send the diagnostics and/or predictions directly to the robotic mobile device 34, and the built-in algorithm (e.g., stored in the memory 136) may automatically (e.g., without receiving control signal from the controller 82) update instructions to obtain information and collect data from specific locations.

FIG. 4 is a flow chart illustrating a method 160 for utilizing the robotic mobile device 34. One or more of the steps of the method 160 may be executed by the controller 82 and/or the mobile device 34. The method 160 includes beginning operation of the turbine system 10 (step 162), beginning operation of the robotic mobile device 34 (step 164), autonomously monitoring for hazardous gas leakage as the robotic mobile device 34 surveys inside the enclosure 14 (step 166), and communicating detected parameters and/or presence of detected leak to the controller 82 (step 168). In other embodiments, upon beginning operation of the robotic mobile device 34 (step 164), the method 160 may include receiving instructions form the controller 82 (step 170), monitoring for hazardous gas leakage at a particular location upon detection or prediction of leakage in response to instructions from the controller 82 (step 170), and communicating detected parameters and/or presence of detected leak to the controller 82 (step 172). In particular, upon beginning operation of the turbine system 10, the robotic mobile device 34 also begins its operation to monitor the leakage condition/situation inside the enclosure 14. The robotic mobile device 34 may begin routine surveying as the robotic mobile device 34 moves (e.g., fly) autonomously inside the enclosure 14 to collect information/data, and feed the collected information/data (e.g., one or more parameters) to the controller 82 (step 166). If it was diagnosed, determined or predicted (e.g., base on one or more parameters) that a hazgas leakage has occurred inside the enclosure 14, the controller 82 or the service platform 86 may send a control signal to move the robotic mobile device 14 to the leakage location to monitor the leakage condition.

This written description uses examples to describe the present embodiments, including the best mode, and also to enable any person skilled in the art to practice the presently disclosed embodiments, including making and using any devices or systems and performing any incorporated methods. The patentable scope of the presently disclosed embodiments is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system, comprising:
a gas turbine enclosure; and
a robotic mobile hazardous gas detection device disposed within the gas turbine enclosure, wherein the robotic mobile hazardous gas detection device comprises one or more sensors configured to detect one or more parameters related to hazardous gas leakage within the gas turbine enclosure, and the robotic mobile hazardous gas detection device is configured to move to different locations within the gas turbine enclosure to monitor for hazardous gas leakage.

2. The system of claim 1, wherein a gas turbine engine is disposed in the gas turbine enclosure.

3. The system of claim 1, wherein the robotic mobile hazardous gas detection device is configured to fly to the different locations within the gas turbine enclosure.

4. The system of claim 1, wherein the robotic mobile hazardous gas detection device is configured to autonomously move to the different locations within the gas turbine enclosure to monitor for hazardous gas leakage.

5. The system of claim 4, wherein the robotic mobile hazardous gas detection device comprises a memory and a processor configured to execute instructions stored on the memory, and the robotic mobile hazardous gas detection device is configured to utilize an algorithm or model stored on the memory to map and navigate the gas turbine enclosure.

6. The system of claim 4, wherein the robotic mobile hazardous gas detection device is configured to move to a particular location based on the one or more parameters detected by the one or more sensors.

7. The system of claim 1, wherein the robotic mobile hazardous gas detection device comprises a transmitter configured to wirelessly transmit the one or more parameters detected by the one or more sensors to a controller disposed outside of the gas turbine enclosure.

8. The system of claim 1, wherein the robotic mobile hazardous gas detection device comprises a receiver configured to wirelessly receive information related to monitoring for hazardous gas leakage from a controller disposed outside of the gas turbine enclosure.

9. The system of claim 8, wherein the robotic mobile hazardous gas detection device is configured to receive, via the receiver, a control signal that causes the robotic mobile hazardous gas detection device to move to a particular location within the gas turbine enclosure to monitor for hazardous gas leakage.

10. The system of claim 1, wherein the one or more sensors comprise a microstructure gas sensor, infrared point sensor, calorimetric thermoelectric gas sensor, ultrasonic sensors, or surface acoustic wave sensor.

11. A robotic mobile hazardous gas detection device, comprising:
one or more sensors configured to detect one or more parameters related to hazardous gas leakage within a gas turbine enclosure;
a transmitter configured to wirelessly transmit the one or more parameters detected by the one or more sensors to a controller disposed outside of the gas turbine enclosure; and
a receiver configured to wirelessly receive information related to monitoring for hazardous gas leakage from the controller;
wherein the robotic mobile hazardous gas detection device is configured to fly to different locations within the gas turbine enclosure having a gas turbine engine and to monitor for hazardous gas leakage.

12. The device of claim 11, wherein the robotic mobile hazardous gas detection device is configured to receive, via the receiver, a control signal that causes the robotic mobile hazardous gas detection device to move to a particular location within the gas turbine enclosure to monitor for hazardous gas leakage.

13. The device of claim 11, wherein the robotic mobile hazardous gas detection device is configured to autonomously move to the different locations within the gas turbine enclosure to monitor for hazardous gas leakage.

14. The device of claim 13, comprising a memory and a processor configured to execute instructions stored on the memory, and the robotic mobile hazardous gas detection device is configured to utilize an algorithm or model stored on the memory to map and navigate the gas turbine enclosure.

15. The device of claim 13, wherein the robotic mobile hazardous gas detection device is configured to move to a particular location based on the one or more parameters detected by the one or more sensors.

16. The device of claim 11, wherein the one or more sensors comprise a microstructure gas sensor, infrared point sensor, calorimetric thermoelectric gas sensor, ultrasonic sensors, or surface acoustic wave sensor.

17. A robotic mobile hazardous gas detection device, comprising:
one or more sensors configured to detect one or more parameters related to hazardous gas leakage within a gas turbine enclosure;
a memory; and
a processor configured to execute instructions stored on the memory that cause the robotic mobile hazardous gas detection device to determine if leakage of hazardous gas is occurring within a gas turbine enclosure based on the one or more parameters related to hazardous gas leakage;
wherein the robotic mobile hazardous gas detection device is configured to fly to different locations within the gas turbine enclosure having a gas turbine engine and to monitor for hazardous gas leakage.

18. The device of claim 17, wherein an algorithm or model is stored on the memory that when utilized by the processor enables the robotic mobile hazardous gas detection device to map and navigate the gas turbine enclosure.

19. The device of claim 17, comprising a transceiver, wherein the robotic mobile hazardous gas detection device is configured to wirelessly communicate with a controller disposed outside of the gas turbine enclosure if a hazardous gas leak occurs within the gas turbine enclosure.

20. The device of claim 17, wherein the robotic mobile hazardous gas detection device is configured to autonomously move to the different locations within the gas turbine enclosure to monitor for hazardous gas leakage.

21. The device of claim 17, comprising a receiver configured to wirelessly receive information related to monitoring for hazardous gas leakage from a controller disposed outside the gas turbine enclosure, wherein the robotic the robotic mobile hazardous gas detection device is configured to receive, via the receiver, a control signal that causes the robotic mobile hazardous gas detection device to move to a particular location within the gas turbine enclosure to monitor for hazardous gas leakage.
